(19) United States
(12) Patent Application Publication
(10) Pub. No.: US 2024/0099829 A1
(43) Pub. Date: Mar. 28, 2024

(54) METHOD OF LIGAMENT REPAIR AND AUGMENTATION

(71) Applicants: Smith & Nephew, Inc., Memphis, TN (US); Smith & Nephew Orthopaedics AG, Zug (CH); Smith & Nephew Asia Pacific Pte. Limited

(72) Inventors: Zenan Qi, Attleboro, MA (US); Michael Thyden, Billerica, MA (US); Stephen A. Santangelo, Tolland, CT (US); Jon-Paul Rogers, North Smithfield, MA (US); Justin A. Barbas, West Bridgewater, MA (US)

(21) Appl. No.: 18/468,129

(22) Filed: Sep. 15, 2023

Related U.S. Application Data
(60) Provisional application No. 63/408,993, filed on Sep. 22, 2022.

(51) Int. Cl. A61F 2/08; A61B 17/06; A61B 17/11

(52) U.S. Cl. CPC A61F 2/0811; A61B 17/06066; A61B 17/06166; A61B 17/1146; A61F 2002/0852; A61F 2220/0075; A61F 2250/0012

(57) ABSTRACT

Disclosed herein is a method of both repairing a ligament and augmenting the repair. A single anchor may provide a single anchoring location for both a repair member and an augmenting member at a first end of the ligament.

### CROSS-REFERENCE TO RELATED APPLICATIONS

This application claims the benefit of U.S. provisional application Ser. No. 63/408,993 filed Sep. 22, 2022, and titled, “METHODS OF LIGAMENT REPAIR AND AUGMENTATION”. This provisional application is incorporated by reference herein, in its entirety as if reproduced in full below.

### FIELD

The present disclosure relates to methods and systems for repairing and augmenting a ligament.

### BACKGROUND

A ligament repair may be augmented with a secondary construct. Augmenting the ligament repair may improve surgical outcomes. Augmenting may involve placing a flexible augmenting member along the repaired ligament, which may help prevent failure of the repaired, but still fragile ligament as it heals post-surgery. Augmenting may also allow the patient to return to normal activity more quickly. Augmenting preferably guards against overly stressing the repaired ligament, without inhibiting healing of the repair. For example, a flexible augmenting member may limit loading on the repaired ligament when a patient stumbles and slightly twists their ankle. Augmenting may provide secondary or back up support for ligament repair of an articulating joint in the body, including but not limited to a foot/ankle, a knee, a shoulder, or a wrist/hand. Some example ligaments may include the Anterior Talofibular Ligament (ATFL), the Calcaneofibular Ligament (CFL), the Medial Collateral Ligament (MCL), Lateral Collateral Ligament (LCL).

Current techniques and systems for augmentation add an augmenting construct supplemental to the repair construct. For example, after ligament repair, which may include a repair construct with sutures and tissue anchors, the augmenting construct may then be installed. This may add at least two further bone anchors and another flexible member coupled therebetween. The augmenting construct therefore may add time and costs to the procedure. The augmenting construct may also remove additional bone during installation of any bone anchors. In the case of smaller joints where bones are naturally smaller, such as the ankle joint, this additional bone removal may weaken the remaining bone structure. The augmenting construct may increase local tissue reactions concomitant with the increased volume of foreign components. The augmenting construct may increase palpability of the final repair configuration, especially for repair locations close to a patient's skin, which may irritate the local area and also be unnerving for the patient. Therefore, there is a need for a method and system that may address these shortcomings.

As way of a specific example, FIG. 1 illustrates a known repair and augmentation technique. FIG. 1 illustrates an ankle 10, with a repair of an ATFL 12 that may include a repair or reattachment of a superior portion of the CFL 14 as well. The ligaments 12,14 may be repaired by inserting two bone anchors 20, 22 into the fibula 16, (shown as dotted circle). Each bone anchor 20, 22 includes its own repair suture 20a, 22a. The repair sutures 20a, 22a may then be operatively coupled to portions of the ligaments 12, 14, and knotted to complete the repair. (Knots of repair suture 20a, 22b are shown, with the anchors 20, 22 hidden underneath). The ligaments such as the ATFL 12 may then be augmented with augmenting construct 30, including a first augmenting anchor 32, a second augmenting anchor 34 and a flexible augmenting member 36 coupled therebetween. The fibula 16 now includes three anchors (20, 22 and 34) and therefore three portions of the fibula 16 have been removed. The final repaired configuration includes four bone anchors.

### SUMMARY

Described herein are various improvements in methods and systems for repairing and augmenting at least one ligament of a joint. These systems may include a plurality of bone anchors, that may be different from one another. One of the bone anchors may be coupled to or configured to couple to a plurality of flexible members and may provide a single anchoring point for both the repair and augmentation. Bone anchors may be cylindrical rigid anchors, or all-suture anchors. Bone anchors may include a knotless mechanism such as an axially moveable plug or a cinchable splice along a portion of one of the flexible members. At least one of the flexible members may primarily repair the ligament. At least one of the flexible members may primarily augment the ligament repair. At least one of the flexible members may be coupled to or configured to couple to suture passers such as needles or passing instruments. Flexible members may include suture, suture tape, suture mini tape, grafts, bio inductive scaffolds, cable, wire of ribbon.

An example technique for repairing and augmenting a ligament is disclosed herein, including inserting a first anchor into a first bone adjacent a first anatomical attachment footprint of the ligament. A flexible repair member and a flexible augmenting member may be operatively coupled to the first anchor during insertion and may extend from a proximal end of the first anchor. With the first anchor inserted, the ligament is repaired using at least the flexible repair member operatively coupled to the first anchor. With the first anchor inserted the flexible augmenting member is extending across the ligament towards a second bone. A second anchor may be inserted into the second bone adjacent a second anatomical attachment point of the ligament. The flexible augmenting member may be operatively coupled to the second anchor. With the flexible augmenting member operatively coupled to both anchors, tension is adjusted and once a target tension has been achieved the flexible augmenting member is locked at that tension. The locking may occur via a locking member associated with the second anchor.

In some example techniques, the flexible augmenting member may be slidingly coupled around the first anchor such that two flexible augmenting member limbs of the flexible augmenting member extend from the first anchor proximal end. The flexible repair member may include a pre-attached needle, and repairing the ligament may include inserting the flexible repair member through the ligament, aided by the needle. The flexible augmenting member may be passed through the ligament before extending it across the ligament towards the second bone and/or before coupling the flexible augmenting member to the second anchor. Repairing the ligament may cover the first anchor with the ligament. The flexible repair member may be a braided suture, and the flexible augmenting member may be a suture tape, different than the flexible repair member. The first anchor may be an all-suture anchor and wherein inserting includes tensioning at least one of the flexible members extending proximally from the first anchor to deploy and set the first anchor within the first bone. The second anchor may be a knotless anchor.

In some example techniques, the flexible repair member may include a first and second repair suture, each repair suture extending from a proximal end of the first anchor. Repairing the ligament may include determining a central axis location of the ligament augmentation and inserting the first anchor on the central axis location and coupling the first repair suture along the ligament at a laterally offset location from the central axis of the ligament on a first side of the central axis and coupling the second repair suture at another laterally offset location from the central axis, on an opposite side of the central axis. The laterally offset location and the another laterally offset location may be spaced between 5-15 mm apart from each other. The first and second repair sutures may extend through the ligament and then may be drawn across a top surface of the ligament and tied together to repair the ligament in a bridging arrangement.

Some example techniques may also include passing a passing instrument from an exterior outer surface of the ligament through to an inner surface of the ligament, engaging the flexible augmenting member extending from the first anchor with the passing instrument and drawing the flexible augmenting member through the ligament before extending the flexible augmenting member across the ligament towards the second bone. Repairing the ligament may be generally intended to reattach the ligament to the first bone. Passing the passing instrument may occur before repairing the ligament.

Another example method of attaching an ATFL to a fibula to repair the ligament and also augmenting the repaired ligament is disclosed herein. This method includes inserting an all-suture anchor into a distal end of the fibula at a location coincident with a first anatomical attachment footprint of the ATFL to be attached. The all-suture anchor is obtained operatively coupled to a flexible repair member and a flexible augmenting member. The method includes tensioning at least one the flexible repair member and the flexible augmenting member to deploy the all-suture anchor within the fibula. The ATFL is then reattached to the fibula with the flexible repair member. The flexible augmenting member is the passed through a thickness of the ATFL and then over an external surface of the ATFL to a talus bone. The flexible augmenting member is then coupled to the talus.

Another example method of repairing and augmenting an ATFL is disclosed herein, starting with obtaining a soft or all-suture anchor assembly including a single soft anchor body with a first repair suture, a second repair suture and an augmentation suture tape coupled thereto. This assembly is inserted into a first bone at or adjacent a first anatomical attachment footprint of the ATFL. The ATFL is coupled to the single anchor with the first and second repair suture. The augmentation suture tape is passed through the ATFL at a location adjacent the first anatomical attachment footprint and then over the ATFL to a second anchor. The second anchor is inserted into a second bone adjacent a second anatomical attachment footprint of the ATFL before locking the augmentation suture tape with the second anchor without tying a knot.

This example method may include coupling the first repair suture to a first location along the ATFL and coupling the second repair suture to a second, different location along the ATFL.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1 illustrates an existing repair including components for ligament repair and augmentation;

FIGS. 2A-2G illustrate methods and components for repairing and augmenting ligaments of the ankle, in accordance with this disclosure; and

FIG. 2H illustrates another example method for repairing and augmenting a ligament, in accordance with this disclosure.

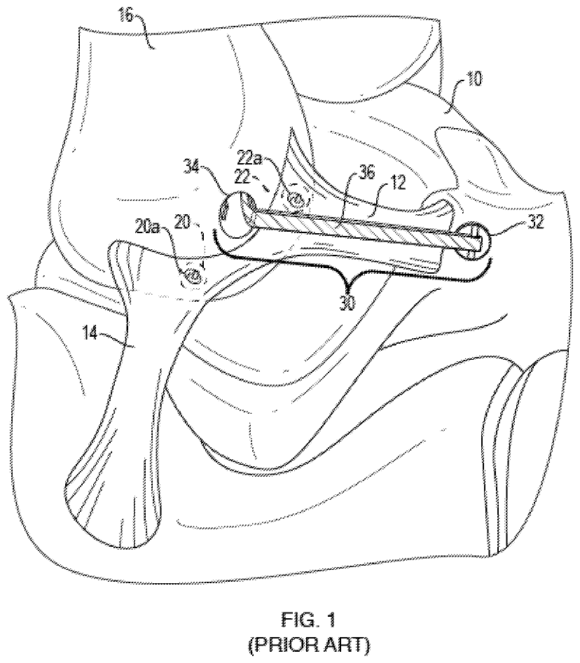

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

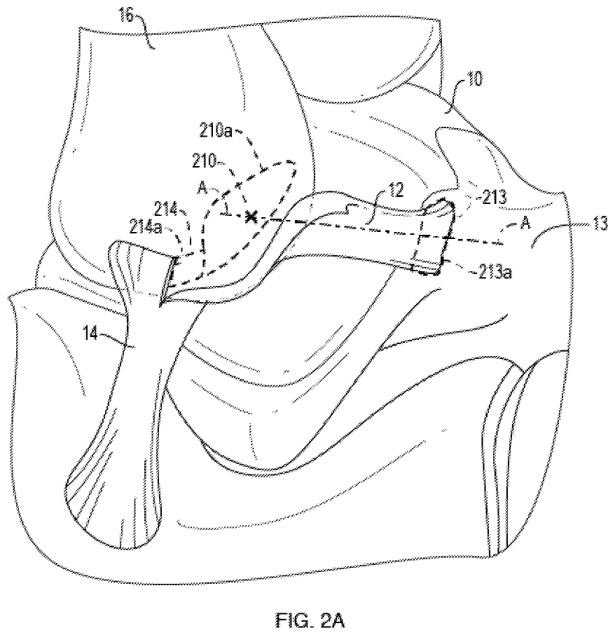

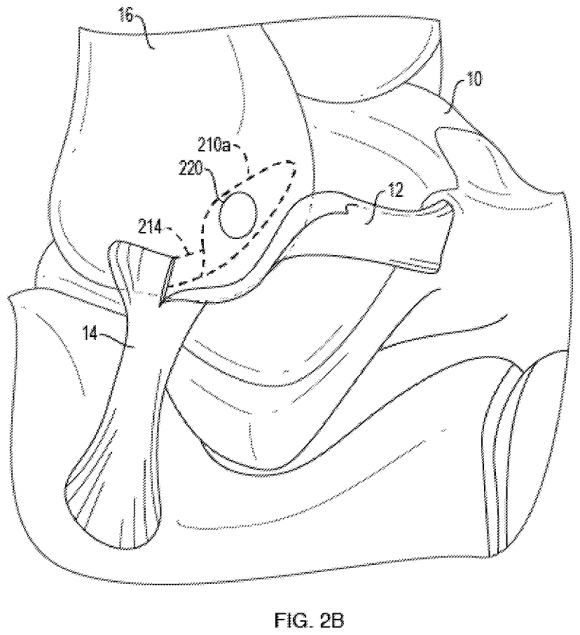

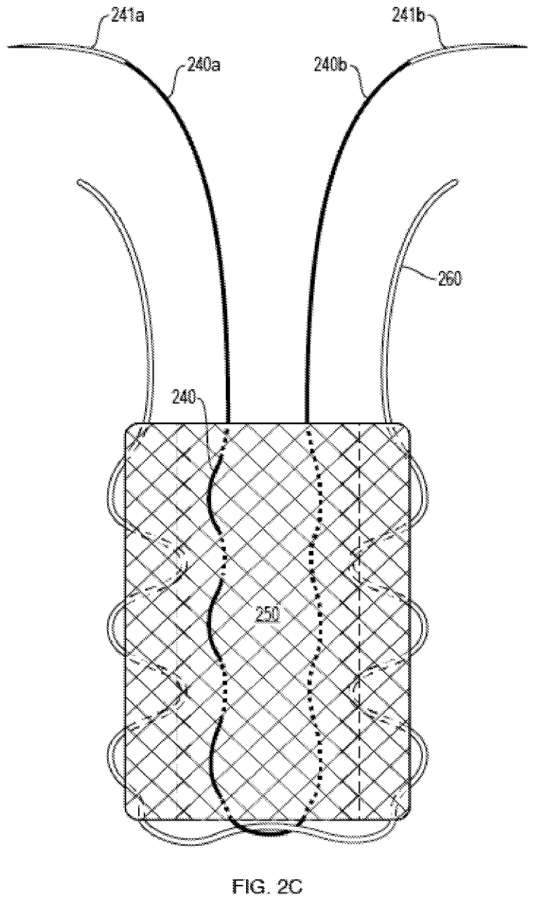

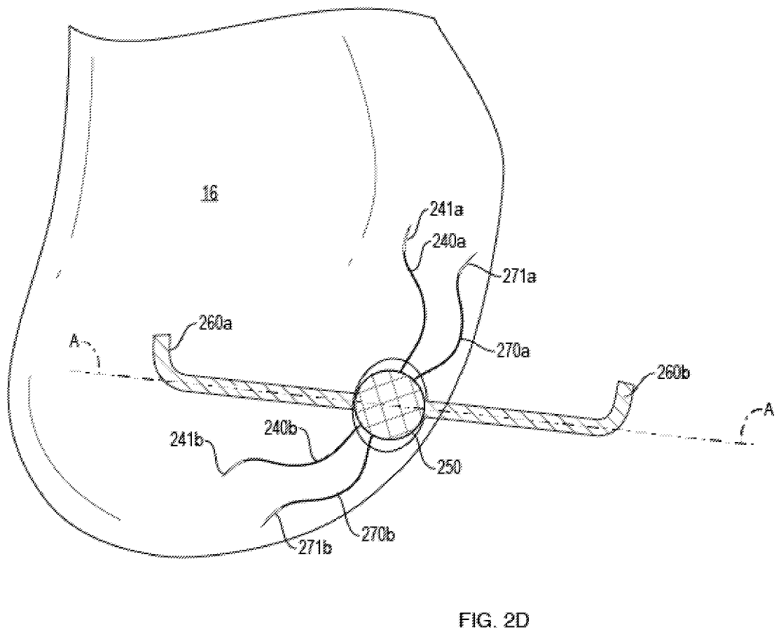

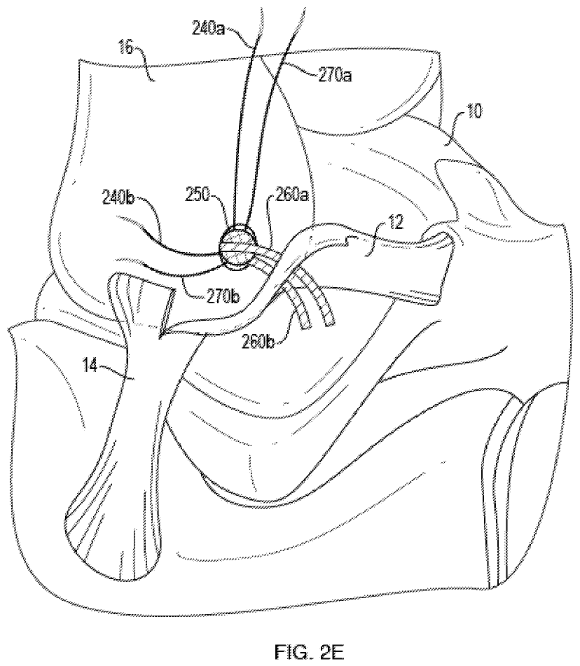

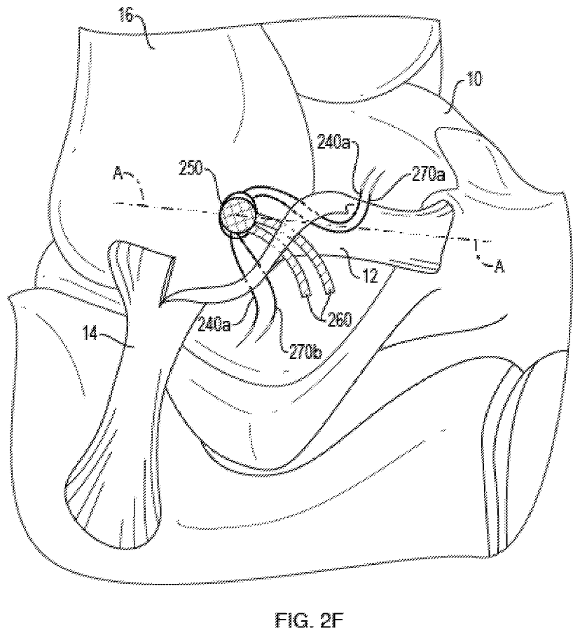

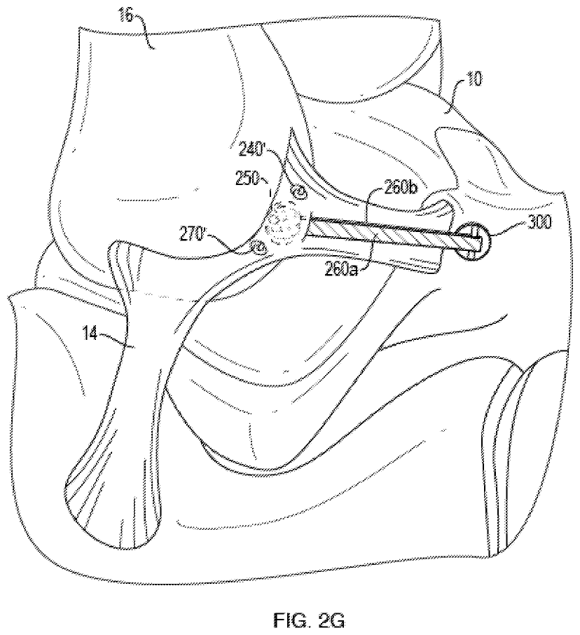

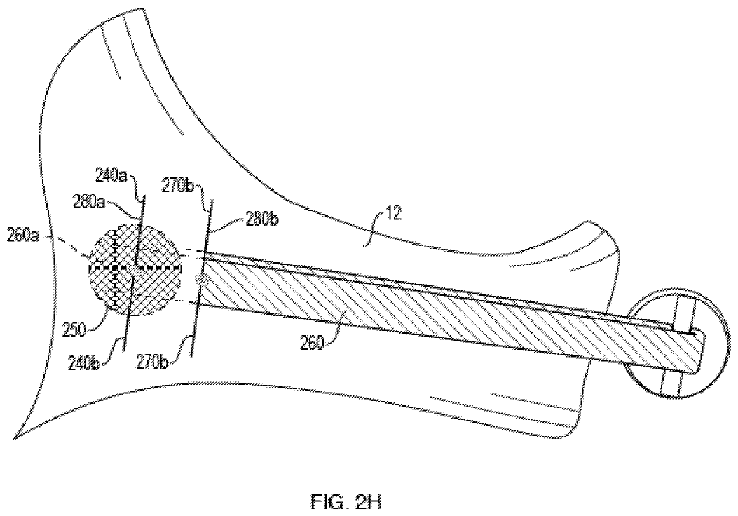

FIG. 2A illustrates a torn ligament of the ankle 10. As shown the proximal end of ATFL 12 has become disconnected from its attachment footprint 210 along the anterior lateral malleolus of the fibula 16. Approximate footprint boundary 210a is illustrated with a broken line. The CFL 14 may also be partially disconnected from its attachment footprint 214 along the distal surface of the fibula 16. Approximate footprint boundary 214a of the disconnected portion is illustrated with a broken line. The ATFL 12, as illustrated, is attached (not torn) at or near the talus 13, defining an attached talus footprint 213, the approximate boundary illustrated with dashed line 213a.

The method may include first determining a center of the talus attachment footprint 213 and an approximate center of the ligament ATFL footprint 210 on the fibula, when re-attached. The approximate center of the footprint 210 is represented by the identifier “X” on FIG. 2A. An axis (A-A) extending through these two centers defining a central or augmenting axis is now defined for later use. In some example methods, the center X of the fibula re-attachment footprint may be modified to include both the ATFL and CFL footprint (210, 214), which may shift the center X distally. In other methods, only a center of the ATFL re-attached footprint 210 may be determined.

Moving on to FIG. 2B, the method may include forming a hole 220 in the fibula 16. Hole 220 may be drilled and may be located along the axis A-A and within footprint 210 of the ATFL. Hole 220 may be formed coincident with location “X” (shown in FIG. 2A). This hole 220 defines the position of the first anchor 250 that may be at a central location relative to the ligament repair and may be under the reattached ligament, once repaired. This may preferably locate the first anchor 250 at or close to the augmenting axis (A-A). First bone anchor 250 may then be placed within the hole 220 and anchored therein. In other methods, a self-tapping bone anchor may be used, such that a bone hole (or a complete bone hole) is not initially prepared before anchor insertion.

First bone anchor 250 may be obtained with at least one flexible repair member and at least one flexible augmenting member already coupled thereto. In other embodiments, bone anchor 250 may be provided with a snare or shuttle (not shown) and the method may include coupling at least one of the flexible members (repair member or augmenting member) to the bone anchor 250 during the procedure. Coupling may occur either before or after insertion of the bone anchor 250 into the bone. This option may allow the surgeon to select both the quantity, type and size of each flexible member (repair member or augmenting member), depending on the degree of repair and augmentation preferred.

In some embodiments, the first bone anchor 250 may be an all-suture anchor (as illustrated in FIG. 2C), provided with at least one repair member 240 and at least one flexible augmenting member 260 coupled thereto. The method may include tensioning at least one of the ends of one of the flexible members (240, 260) coupled to the all-suture anchor to deploy the all-suture anchor within the fibula 16 in hole 220. In other examples, all flexible members that are attached may be tensioned to deploy the all-suture anchor. In some example embodiments, the repair flexible member(s) 240 may include a plurality of members and may be smaller in cross section than the augmenting flexible member 260. Deploying anchor 250 may include only tensioning the augmenting flexible member 260, as this may be the member with the highest tensile strength, and therefore allow the greatest deploying load thereon. For some all-suture anchors, higher deploying tensions may in turn increase fixation strength of the anchor 250 within the bone.

Flexible repair member(s) 240 may have needles pre-attached thereto (not shown). Therefore, in some embodiments these flexible repair member(s) 240 may be preferably isolated from any anchor deployment system to avoid the complication of needle management on deploying members within an insertion instrument. In this particular option, the method may include using the needle to pierce the ligament tissue and thereby couple the bone anchor 250 to the ligament via member(s) 240 and repair/reattached the ligament. The needle(s) (241a, 241b) may then be removed.

An example all-suture anchor that may be first anchor 250, is disclosed in commonly owned U.S. Pat. No. 9,962,149; herein incorporated in its entirety by reference. A flexible repair member 240 may interweave through the anchor braided tubular body such that two limbs 240a, 240b extend from a proximal end thereof. At least one of the limbs 240a may be obtained with a needle 241a, 241b attached, as discussed here. A flexible augmenting member 260 may be provided also attached to the soft anchor 250 and may interweave repeatedly through the anchor at a location circumferentially offset from the repair member 240. In some embodiments a second flexible repair member may be coupled to the anchor, in a similar manner to repair member 240. An example all-suture anchor with needles and insertion instrument system is disclosed in commonly owned PCT patent No. US2021/3459027, filed May 2021, titled “TISSUE REPAIR SYSTEM”, herein incorporated in its entirety by reference.

FIG. 2D illustrates fibula 16 with the first bone anchor 250 inserted into hole 220, the hole walls defined by fibula 16. The ligaments and other portions of the ankle are removed from FIG. 2D for simplicity of understanding. Anchor 250 is shown with two limbs 260a, 260b of augmenting flexible member 260, two limbs 240a, 240b of first repair member 240 and two limbs 270a, 270b of a second repair member 270 extending from hoe entrance. All flexible members (240, 260, 270) may be operatively coupled to the anchor 250 either as obtained or during the procedure, as disclosed herein. It may be preferable to have the augmenting flexible member 260 oriented in a first direction that is along the axis of augmentation (A-A), with the repair members 240, 270 oriented circumferentially offset therefrom. As shown, the repair members 240, 270 are approximately perpendicularly arranged relative to the augmenting member 260. This arrangement is configured to align the flexible members (240, 270) according to their function. For example, the augmenting member 260, in its final configuration extends along the line of augmentation A-A while the repair members (s) (240, 270) may couple to the ligament 12, 14 at locations that are laterally offset from the axis A-A. This arrangement allows the augmenting member 260 to extend along the axis A-A without undue twists that may interfere with smooth augmentation. The method may include aligning markings on the anchor or anchor insertion device to align the augmenting member 260 along axis A-A and the repair members laterally disposed therefrom. Example markings are shown in U.S. application Ser. No. 17/470,509, titled “JOINT REPAIR AUGMENTATION”, commonly owned and herein incorporated by reference, in its entirety.

As shown, repair member 240 may include a first end 240a that extend from a first side of axis A-A, and a second end that extends from a second or opposite side of the axis A-A. In other embodiments the repair member 240 may be interwoven through the anchor 250 along a single side of the anchor 250 and therefore both ends 240a, 240b may extend from the same side anchor relative to axis A-A. This may further arrange the repair member 240 with both ends 240a, 240b closer to a repair location along the ligament (12 or 14. Second repair member 270 may also be similarly arranged.

Once the first anchor 250 is inserted and set (deployed) within the fibula 16 at the determined target location as disclosed, the flexible augmenting member 260 may be passed through from an inner or under surface of the ligament 12 to an outer most surface. Augmenting member 260 may therefore be provided with needles attached to pierce the soft tissue. Augmenting member 260 may be coupled to the first anchor 250 such that two limbs 260a, 260b extend from a proximal end of the first anchor 250. Augmenting member 260 may be slidingly or flossingly coupled to first anchor 250. Each limb 260a, 260b may be passed through a single location (either one at a time or together) through the ligament 12, or each limb 260a, 260b may have its own passage through the ligament, such that first limb 260a extends through a first passage through the ligament 12, and the second limb 260b extends through a second passage, separated from the first passage through the ligament 12. This may further improve the compression of the ATFL 12 to the fibula 16. This may provide two differing vectors of augmentation along the repaired ligament.

Flexible augmenting member 260 may be obtained with a needle pre-attached, and the method may include piercing the ligament 12 with the needle to pass the augmenting member 260 through the ligament 12. In other example methods, where a needle is not provided or pre-attached, a needle may be coupled to the augmenting member 260 before passing at least one limb of the augmenting member 260 through the ligament 12. In another example method, a suture passer may form a passage through the ligament 12 and draw the augmenting member 260 (including limbs 260a, 260b) through the ligament 12. A suture passer with a piercing tip may be preferable for use with this method, such as a suture shuttle device disclosed in U.S. Pat. No. 10,265,062, titled “Surgical instrument for manipulating and passing suture”, commonly owned and herein incorporated by reference in its entirety.

In other example embodiments, flexible augmenting member 260 may have a first limb end fixedly coupled within the anchor 250 such that only a single limb (260a) of the augmenting member 260 extends from a proximal end of the anchor 250. Augmenting flexible member 260 may include a stopper knot for example to an end of the augmenting member 260 with the anchor 250. In this example only a single length of augmenting flexible member (260a) may provide augmentation.

Before passing the flexible augmenting member limbs 260a, 260b, a location through the ligament may be first determined. The ligament 12 may be temporarily placed over fibula 16 along the footprint 210, 214, and also over the first anchor 250. Augmenting member 260 may preferably be passed through the ligament 12 to the ligament exterior surface at a location that lies directly over the anchor 250. In some other examples methods, augmenting member 260 may be passed through the ligament 12 at a location along the augmentation axis A-A and spaced axially away, along axis A-A from anchor 250.

Once the augmenting member 260 is passed through the ligament 12, the ligament 12 may then be re-attached using at least one repair member 240, and in some cases two repair members 240, 270. The augmenting flexible member 260 may be passed but still loose to allow the ligament footprint to be visualized while arranging the repair member 240 or members 240, 270. Should the repair have a small footprint, only a single repair member 240 may be required. A preferred location for coupling the repair member 240 to the ligament is determined. This may be laterally offset from a passing location of the augmenting flexible member 260. This repair location may be laterally offset from the augmenting axis A-A. The repair member 240 may couple to the ligament 12 at a location 5 mm offset from the first anchor 250 either further anteriorly or further distally along the fibula 16, depending on the arrangement of the tear. Two limbs 240a, 240b may be passed through the ATFL 12, adjacent each other using pre-attached needles 241a, 241b before removing the needles 241a, 241b. The ligaments 12, 14 may then be abutted to the fibula 16 and over the anchor 250 at the determined attachment footprint location and then a knot 240′ tied to secure the repair member 240 and thereby at least one ligament (12, 14) to the fibula 16. At least one of the ligaments 12, 14 may now cover the first bone anchor 250, and by repaired, while the augmenting flexible member 260 is loosely extending through the ligament 12.

For ligament tears that are larger in extent, two repair members 240, 270 may be coupled to the first anchor as obtained or coupled to the first anchor 250 during the procedure, as disclosed herein. These two repair sutures (240 and 270) may be coupled to the ligament at two different locations offset from each other. This is illustrated in FIG. 2F and also FIG. 2G, where two repair members have formed a repair knot 240′, 270′. It is not necessary that the same suture be used to form each repair knot (240′, 270′). For example, ends 240a and 270a (with are ends of separately formed flexible members) may define the repair a first location of the ligament and be tied together; i.e knot 240′ could be formed with ends 240a and 270a. Similarly ends 240b and 270b (with are ends of separately formed flexible members) may define the repair another location along the ligament and be tied together; i.e knot 270′ could be formed with ends 240b and 270b.

In another example methods, each limb of each repair member (240a, 240b, 270a, 270b) may be coupled to each other in a bridging fashion (FIG. 2H). The bridge(s) 280a, 280b may extend under or over the augmenting member 260. It may be preferable to extend the bridge(s) 280a, 280 under the augmenting member 260, to reduce interference with augmentation. Bridges may be coupled via knots, or knotlessly using finger traps.

A second anchor 300 may couple to the augmenting flexible member 260 and fix the augmenting member 260 to the talus, after a target tension is achieved. Second anchor 300 may be a knotless anchor. Second anchor 300 may be inserted first and the augmenting member 260 coupled thereto after the second anchor is in place. Second anchor 300 may be inserted at a location along the axis A-A but outside of the ligament attachment footprint boundary 213 on the Talus.

In some methods, with extensive detachment of the CFL 14, a third anchor may be disposed on the fibula distal surface nearer the CFL 14 and the ligament 14 reattached via this third anchor. In another method, the third anchor may also include both a repair member and augmenting member, and the CFL 14 may also be both repaired and augmented in a similar fashion to the method of ATFL repair disclosed herein.

In other example method, the anchor 250 may be placed at allocation that is offset from X and laterally from axis A-A. This location may be chosen to move the anchor 250 to a location closer to a preferred location for the repair suture. The augmenting member 260 may then extend at an angular offset from axis A-A, while the repair suture may couple to the ligament 12 directly over anchor 250. This may provide a stronger repair with a slight offset in Augmentation.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

What is claimed is:

1. A method of repairing and augmenting a ligament, comprising:
inserting a first anchor into a first bone adjacent a first anatomical attachment footprint of the ligament, a flexible repair member and a flexible augmenting member operatively coupled to the first anchor and extending from the first anchor; and
with the first anchor inserted;
repairing the ligament with the flexible repair member;
extending the flexible augmenting member across the ligament towards a second bone;
inserting a second anchor into the second bone adjacent a second anatomical attachment point of the ligament;
operatively coupling the flexible augmenting member to the second anchor and adjusting tension on the flexible augmenting member that extends between the first and second anchor; and
once a target tension has been achieved locking the flexible augmenting member with the second anchor.

2. The method of claim 1 wherein the flexible augmenting member is slidingly coupled around the first anchor such that two flexible augmenting member limbs of the flexible augmenting member extend from the first anchor proximal end.

3. The method of claim 1 wherein the flexible repair member includes a needle attached thereto, and wherein repairing the ligament including inserting the flexible repair member with the needle through the ligament.

4. The method of claim 1 further comprising passing the flexible augmenting member through the ligament before extending it across the ligament towards the second bone.

5. The method of claim 1 wherein repairing the ligament covers the first anchor.

6. The method of claim 1 wherein the flexible repair member includes a first and second repair suture, each repair suture extending from a proximal end of the first anchor, and wherein repairing the ligament includes;
determining a central axis location of the ligament augmentation;
inserting the first anchor on the central axis location; and
coupling the first repair suture along the ligament at a laterally offset location from the central axis of the ligament on a first side of the central axis and coupling the second repair suture at another laterally offset location from the central axis, on an opposite side of the central axis.

7. The method of claim 6 wherein the laterally offset and the another laterally offset location are spaced between 5-15 mm apart.

8. The method of claim 6 wherein the first and second repair sutures extend through the ligament and then are drawn across a top surface of the ligament and tied together to repair the ligament in a bridging arrangement.

9. The method of claim 1 wherein the flexible repair member is a suture, and the flexible augmenting member is suture tape.

10. The method of claim 1 wherein the first anchor is an all-suture anchor and wherein inserting includes tensioning at least one of the flexible members extending proximally from the first anchor to deploy and anchor the first anchor with the first bone.

11. The method of claim 1 further comprising passing a passing instrument from an exterior outer surface of the ligament through to an inner surface of the ligament and engaging the flexible augmenting member extending from the first anchor and drawing the flexible augmenting member through the ligament before extending the flexible augmenting member across the ligament towards the second bone.

12. The method of claim 11 wherein repairing the ligament reattaches the ligament to the first bone and wherein the passing instrument is passed before repairing the ligament.

13. The method of claim 1 wherein the second anchor is a knotless anchor.

14. A method of attaching an ATFL to a fibula and thereby repairing the ligament and augmenting the repaired ligament, comprising:
inserting an all-suture anchor into a distal end of the fibula at a location on a first anatomical attachment footprint of the ATFL to be attached, the all-suture anchor operatively coupled to a flexible repair member and a flexible augmenting member;
tensioning at least one the flexible repair member and the flexible augmenting member to deploy the all-suture anchor within the fibula;
reattaching the ATFL to the fibula with the flexible repair member;
passing the flexible augmenting member coupled to the all-suture anchor through a thickness of the ATFL and then over an external surface of the ATFL to a talus bone; and
coupling the flexible augmenting member to the talus.

15. A method of repairing and augmenting an ATF ligament comprising:
obtaining a soft anchor assembly including a single soft anchor body with a first repair suture, a second repair suture and an augmentation suture tape coupled thereto and inserting the soft anchor assembly into a first bone at or adjacent a first anatomical attachment footprint of the ATF ligament;
coupling the ATF ligament to the single anchor with the first and second repair suture;
extending the augmentation suture tape through the ATF ligament at a location adjacent the first anatomical attachment footprint and then over the ATF ligament to a second anchor;
inserting the second anchor into a second bone adjacent a second anatomical attachment footprint of the ATF ligament; and
knotlessly locking the augmentation suture tape with the second anchor.

16. The method of claim 15 wherein coupling includes coupling the first repair suture to a first location along the ATF ligament and coupling the second repair suture to a second, different location along the ATF ligament.
